(19) United States
(12) Patent Application Publication
(10) Pub. No.: US 2026/0174355 A1
(43) Pub. Date: Jun. 25, 2026

(54) PERISTOMAL SKIN BARRIER SENSOR

(71) Applicant: Hollister Incorporated, Libertyville, IL (US)

(72) Inventors: Mark W. Jockel, Vernon Hills, IL (US); Adrian P. DEFANTE, Chicago, IL (US); Jonathan Li Chen, Evanston, IL (US); Gracia Cosenza, Evanston, IL (US); Huma Zulfiqar Khan, Corona, CA (US); Aymen Lamsahel, Chicago, IL (US); Madison Russo, Evanston, IL (US); Wala Siddig, Iowa City, IA (US); Ross Wojcik, Overland Park, KS (US)

(73) Assignee: Hollister Incorporated, Libertyville, IL (US)

(21) Appl. No.: 19/126,713

(22) PCT Filed: Dec. 22, 2023
(86) PCT No.: PCT/US2023/085652

§ 371 Date: May 2, 2025

Related U.S. Application Data
(60) Provisional application No. 63/435,490, filed on Dec. 27, 2022.

(51) Int. Cl. A61B 5/103; A61B 5/00; A61B 5/145

(52) U.S. Cl. CPC A61B 5/103; A61B 5/14532; A61B 5/6833

(57) ABSTRACT

A peristomal skin barrier sensor includes a barrier (2) and an internal measurement unit (IMU). The internal measurement unit can be one or more flex sensors (24), an electrical resistance mesh, an e-textile that includes one or more piezoelectric displacement sensors or markers, or an electrical resistance mesh. The peristomal skin barrier sensor is configured to measure a deformation of the barrier.

### BACKGROUND

This disclosure is related to peristomal skin barrier sensors, and more particularly to peristomal skin barrier sensors to sense the movement of the barrier and thus the integrity of the seal between the skin barrier and the peristomal skin.

Referring briefly to FIG. 1, an ostomy is a surgical procedure that reroutes parts of the digestive or urinary system to a stoma 1—an opening in the abdomen through which waste can pass and be collected in a pouch. An ostomy is a critical and often life-saving procedure. An adhesive barrier 2 attaches the pouch 3 to the skin around the stoma (the peristomal skin) to enable waste to be collected in the pouch.

The adhesive on the back side of barriers (the skin side of barriers) varies. The back or skin side of barriers can vary in the shape and surface area of the adhesive. In some barriers the back is fully made from a hydrocolloid adhesive. In other barriers, the hydrocolloid adhesive may be present in a central circular area, but a more band-aid-like tape material may be present around the edges of the barrier.

In an ideal scenario the adhesive barrier and skin would move in unison and the barrier would remain firmly applied to the skin and prevent leakage and irritation to the skin. However, under certain daily activities, the barrier detaches from the skin due to the magnitude and direction of the shear and normal stresses that are induced by various movements, which can differ based on the intensity, frequency, and longevity of these activities, and sometimes due to significant perspiration. The progression of the movement of the adhesive barrier throughout a three-day period is shown in FIG. 2A-2C.

The adhesive barrier currently moves and detaches from the skin, contributing to a leakage of bodily waste and/or complete separation of the pouch from the abdomen. This movement could also cause deformation, irritation, and redness in the skin surrounding the stoma, as shown in FIGS. 3A and 3B.

Currently there are no available methods to measure the movement of the adhesive barrier. This information is necessary to gain an understanding of the changes the barrier undergoes in order to create a stronger adhesive that better complements the skin.

Accordingly, it is desirable to provide a system that senses the deformation and movement of the adhesive barrier.

### BRIEF SUMMARY

A peristomal skin barrier sensor is provided according to various embodiments. In an embodiment, the peristomal skin barrier sensor includes a barrier, an internal measurement unit (IMU) and a flex sensor. The flex sensor is configured to measure a deformation of the barrier.

The flex sensor can be a series of flex sensors. The series of flex sensors can be chained to adjacent ones of the flex sensors. Data is collected at each of the flex sensors of the series of flex sensors. The data collected is voltage data. The flex sensor is configured to test a structure of the barrier.

In embodiments the skin barrier sensor includes a barrier, and an electrical resistance mesh. The electrical resistance mesh is configured to measure a resistance used to generate a 3D surface model. In embodiments the sensor includes a waterproof silicone layer. The waterproof silicone layer can be a first waterproof silicone layer and the electrical resistance mesh can be sandwiched between the first waterproof silicone layer and a second waterproof silicone layer. One of the first and second silicone waterproof layers is positioned adjacent the barrier. The electrical resistance mesh is configured to test a structure of the barrier.

In still another embodiment, the peristomal skin barrier sensor includes a barrier, and an e-textile measurement system. The e-textile measurement system includes an e-textile and one or more piezoelectric displacement sensors configured to measure a deformation of the barrier. The sensor can include a silicon waterproof layer. The sensor can further include an accelerometer on the e-textile. The accelerometer can trigger the one or more piezoelectric displacement sensors. The e-textile measurement system is configured to test a structure of the barrier. In embodiments, the e-textile can be positioned between the silicone waterproof layer and the barrier.

In still other embodiments, a peristomal skin barrier sensor includes a barrier, a water-resistant polymer coating, and an e-textile. The e-textile can include displacement markers configured to measure a deformation of the barrier. In embodiments, the e-textile is positioned between the barrier and the water-resistant polymer coating. The water resistant polymer coating can be a first water resistant polymer coating and the sensor can further include a second water resistant polymer coating such that the e-textile is positioned between the first and second water resistant polymer coatings. The water-resistant polymer coatings can be spray-on coatings. The e-textile is configured to test a structure of the barrier.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 illustrates an ostomy barrier mounted to a user's skin and a two-piece ostomy appliance;

FIG. 2 illustrates the progression of the movement of the adhesive barrier throughout a three-day period;

FIGS. 3A and 3B illustrate a stoma with minor skin deformation (FIG. 3A), and with major skin deformation (FIG. 3B);

FIG. 4 is a functional tree diagram for a method of assessing the performance of an adhesive barrier;

FIG. 5 is a portion of a functional tree diagram for a method of assessing adhesive barrier performance using flex sensors;

FIGS. 6A and 6B are illustrations of flex sensors and sensor mounting;

FIG. 7 illustrates is a 3D printed testbench with radius of 94 mm;

FIG. 8 illustrates the flow of data and energy in a flex sensor embodiment;

FIGS. 9A and 9B are modeled cantilever beams of an adhesive barrier according to embodiments showing a force at the end of the beam with a rigid center of the barrier as the support and the deformable part as the beam;

FIG. 10 is a model of a series of smaller curves defining a single flex sensor;

FIGS. 11A-11D are illustrations of concepts for sensing the movement of the barrier, including an optical solution (FIG. 11A), a laser point tracking solution (FIG. 11B), a motion tracking solution (FIG. 11C), and a sensor system (FIG. 11D);

FIG. 12 is an electrical schematic for an embodiment of a piezoelectric sensor arrangement;

FIG. 13 is an electrical schematic for an embodiment of a force sensing resistor arrangement;

FIG. 14 is an electrical schematic for an embodiment of a flex sensor arrangement;

FIG. 15 is an electrical schematic for an embodiment of a conductive sheet sensor arrangement;

FIG. 16 illustrates the test set up for the conductive sheet sensor arrangement;

FIG. 17 illustrates the relationship between theoretical strain and measured voltage;

FIGS. 18A and 18B illustrate the test set up for the sensor in a cantilever configuration with a rigid support in which FIG. 18A illustrates the beam without a weight, and FIG. 18B illustrates the beam with a weight;

FIG. 19 is a graphical illustration of the test results illustrating the relationship between the load and the flex sensor voltage reading;

FIG. 20 is a graphical illustration of the test results illustrating the relationship between the load and deflection in mm;

FIG. 21 is a graphical illustration of the aggregated data for an average of three trials illustrating the relationship between the deflection and load;

FIG. 22 is a graphical illustration of the relationship between sensor voltage and applied force;

FIG. 23 illustrates the radius of the flex sensor as estimated at a certain distance from the neutral axis by using the strain, in which p represents the radius at that point in the beam and y represents the neutral axis;

FIG. 24 illustrates flex sensors as they can be chained end to end and showing in FIG. 24 flex sensors that are sufficiently small that each of the sensors bends as a simple shape that a cantilever model can predict;

FIG. 25 illustrates a series of flex sensors in a chain and that the connection between flex sensors are tangent to one another;

FIG. 26 is an electrical schematic diagram for the critical system prototype;

FIG. 27 illustrates the sensor the test set up for testing the flex sensor wrapped around a test bench;

FIG. 28 is graphical illustration of a comparison of the actual vs. predicted radius of curvature for various radii;

FIG. 29 illustrates another embodiment of a peristomal skin barrier sensor in which an inertial measurement unit embedded in an e-textile with flex sensors in the textile;

FIG. 30 illustrates still another embodiment of a peristomal skin barrier sensor in which an electrical resistance mesh sandwich has an electrical resistance mesh/strain gauge sandwiched between waterproofing silicone layers and positioned on an adhesive barrier;

FIG. 31 illustrates yet another embodiment of a peristomal skin barrier sensor in which an e-textile has a piezo mesh with piezo displacement sensors and one or more accelerometers to trigger sensing;

FIG. 32 illustrates still another embodiment of a peristomal skin barrier sensor in which an e-textile includes an inertial measurement unit with small thin magnets and an epoxy coating layer on the e-textile, and in which the adhesive barrier includes small thin magnets to maintain the e-textile on the adhesive barrier; and

FIG. 33 illustrates yet another embodiment in which the e-textile has displacement markers sandwiched between water resistant polymer coating layers and a glue adhesive between the water resistant polymer coating and the adhesive barrier.

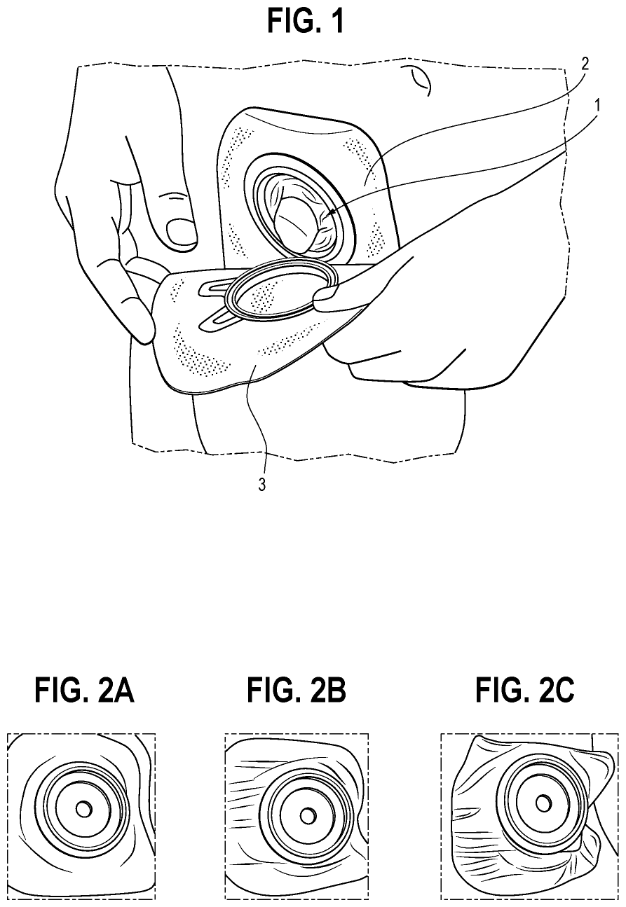

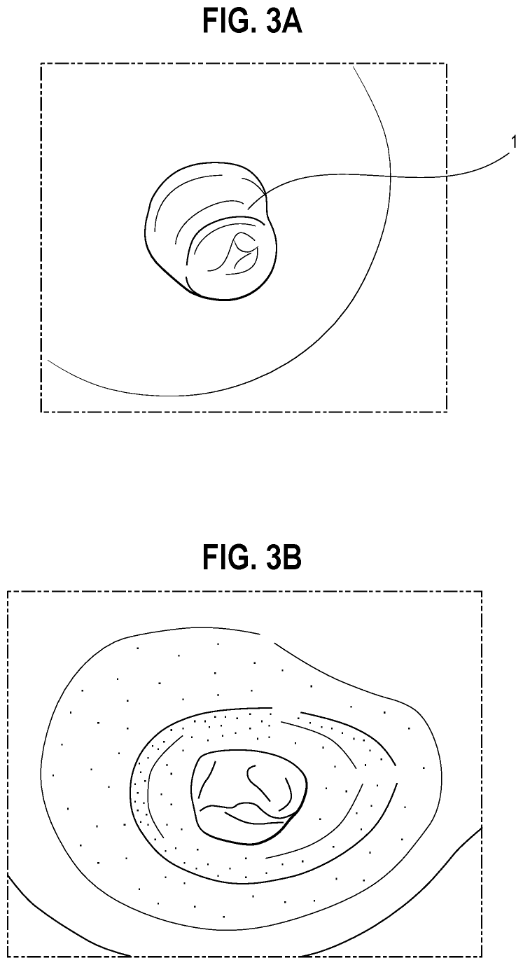

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

The present disclosure includes devices that can monitor and assess the movement of an ostomy skin barrier and thus the mechanical integrity of the skin barrier by monitoring the mechanical deformations the barrier undergoes during use. In an embodiment, the device measures the deformations that the barrier experiences and tracks the barrier's movement on the body over time throughout different activities. As the barrier experiences various types of forces, the device facilitates observation of the different types of barrier deformation and the effect that deformation has on the adhesive and thus the barrier integrity.

The requirements for a sensor device/system were identified as, but not limited to: the ability to accurately monitor deformation of a flexible surface; that it is usable on most if not all ostomy pouch systems; that it is safe and does not cause harm (and preferably not irritation) when worn by a user; that it does not interfere with the functions and performance of the ostomy pouch system; that it withstands everyday functions of the ostomate; that it adapts to the adhesive barrier movement during normal wear and activities; that the system collects data at multiple locations on the adhesive to monitor deformation of the barrier at multiple locations; that the data collected is consistent and reproducible; that the data communication device/method does no interfere with normal movement and activities of the ostomate; and that the data obtained is in the form of mechanical properties, and processes the data into relevant measurements to evaluate adhesive performance.

The requirements for a sensor device/system monitoring metrics were identified as, but not limited to: the total operational period for data collection of single use case of about 5 to about 60 minutes; a sampling frequency of greater than about 1 Hz and preferably at least 10 to draw a balance between useful resolution of data, noise reduction, and ease of data processing; and size adjustability for the stoma over the diameter of the adhesive barrier of at least about 1.0 cm to about 3.0 cm (about 0.4 inches to about 1.2 inches), and preferably about 0.5 cm to about 4.0 cm (about 0.2 inches to about 1.6 inches) based on the adjustability of the barrier.

Additional requirements for the sensor device/system monitoring metrics include water resistance testing, resistance to humidity and small amount of stroma effluent; no significant difference in skin irritation with or without sensor; Young's modulus of no more than about 200 and preferably about 20 for the sensor similar to adhesive barrier and or skin under a tensile test; a maximum load of the ostomy pouch of more than about 0.2 kg to about 0.4 kg (about 0.45 lbs. to about 0.9 lbs.) and preferably more than about 0.5 kg (1.1 lbs.); a minimum number of data collections sites per square centimeter (cm²) of greater than about 10 and preferably greater than about 30 (greater than about 65 sites per square inch and preferably greater than about 194 sites per square inch); that the deformation data is captured in displacement, strain, force, and radius of curvature; that the data is reproducible; that there is no significant difference in performance of activity with our without the device; and that the data produced is consistent with theoretical predictions when deformed around a known curvature, the curvature having a R² or goodness of fit between measured and predicted curvature of greater than about 0.7 and preferably greater than about 0.95.

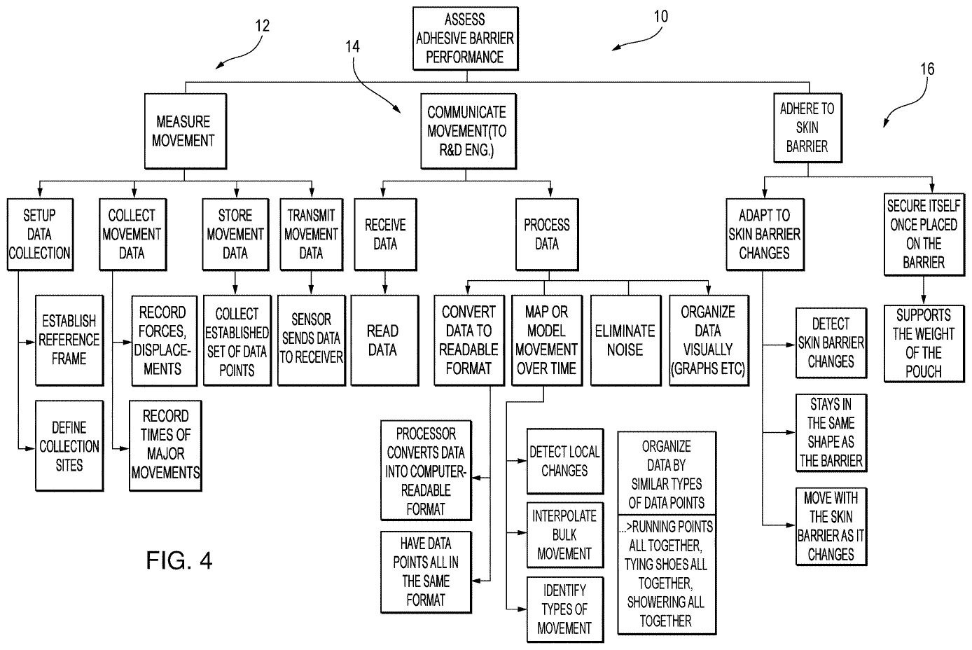

Referring now to FIG. 4, there is shown a functional tree diagram 10 for a method of assessing the performance of an adhesive barrier. Three principle functions are identified, namely, measure barrier movement 12, communicate the measured movement 14 and adherence to the skin barrier 16. In one system of monitoring and assessing the movement of an ostomy skin barrier, the critical system is measuring barrier movement 12, which correlates to assessment of the adhesive barrier performance.

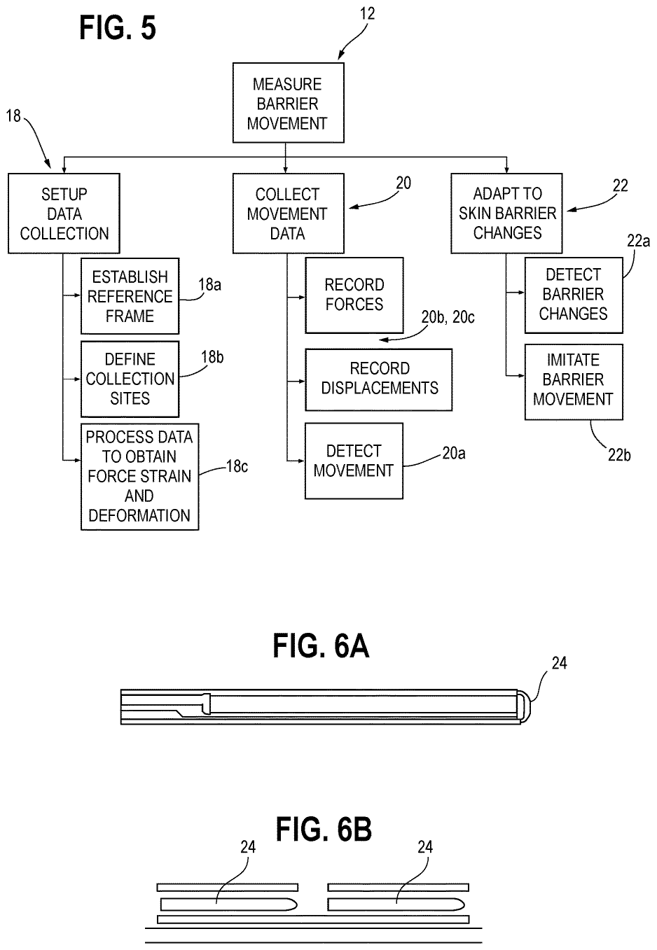

Referring now to FIG. 5, within the critical system there are three sub-functions. The first sub-function is to establish data collection 18 which, in an embodiment, includes establishing a reference frame 18a, defining collection sites 18b, and establishing methodology 18c to process the data collected into force, stress, strain, and deformation. The second sub-function 20 is to collect movement data, e.g., detect movement 20a and record data like forces and deformations 20b, 20c. The third sub-function 22 is for the system to adapt to skin barrier changes, e.g., the system detects changes in the barrier 22a and imitates its movement 22b.

A prototype for the measure barrier movement function was developed having two parts: the circuitry including the sensor 24, i.e., hardware, and the mathematical model used to process data.

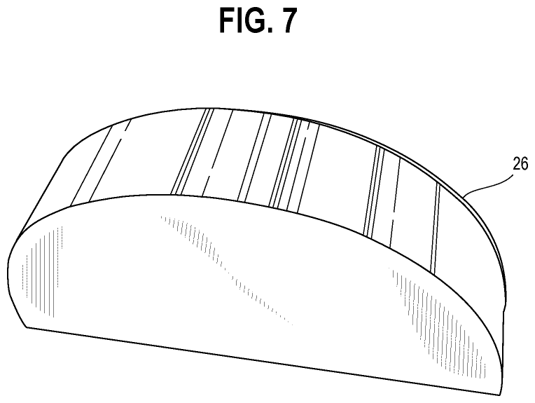

To measure deformation, flex sensors 24 were used. The prototype includes two flex sensors 24 mounted end to end (see, FIG. 6B) and secure to one another to allow the sensor to flex relative to one another. The flex sensors 24 were mounted to a test bench 26 (see FIG. 7), and tape was used to facilitate better conforming the sensors 24 to the test bench. The radius of the test bench was calculated noting that the flex sensor 24 cannot be bent 90° or more without failure.

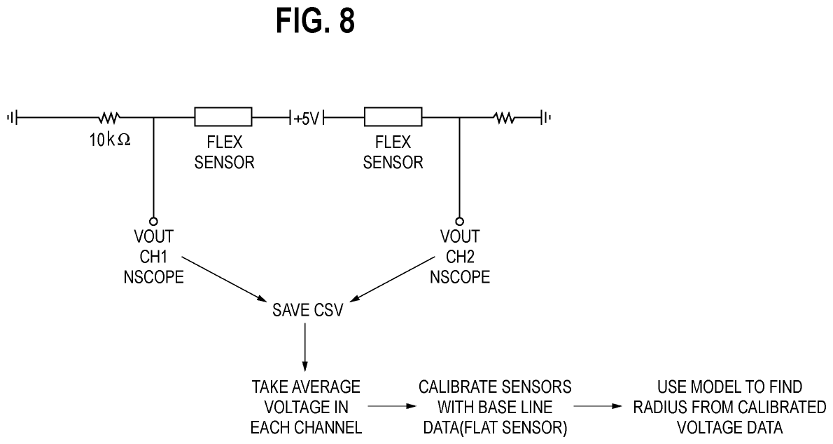

Voltage dividers were used with the flex sensors 24 to better detect voltage changes due to bending and to avoid damaging the flex sensor 24. The voltage was recorded through nScope and saved as a csv file. The average voltage for each channel was then calculated in the csv and processed to find the radius of curvature of the surface. The flow of data and energy is shown in FIG. 8.

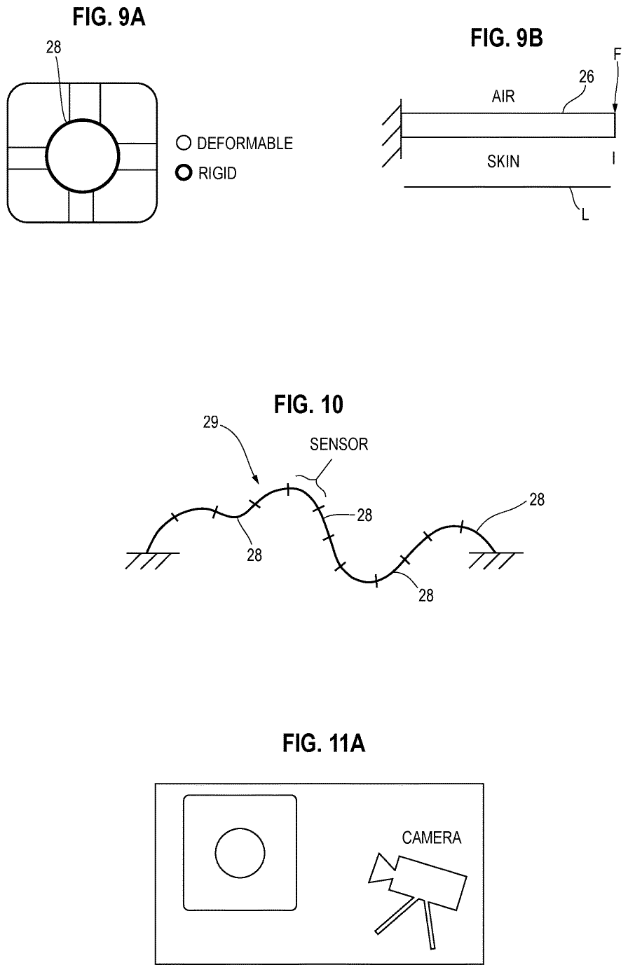

A mathematical model was created to process data collected by the flex sensors 24. The mathematical model converts voltage data obtained from the sensor 24 into force and deformation. It was assumed that the adhesive barrier could be modeled as a set of cantilever beams 26 with a force at the end with the rigid center of the barrier 28 as the support and the deformable part as the beam 26, as illustrated in FIGS. 9A and 9B. Since the adhesive barrier doesn't behave like a rigid beam, the assumption was made that the bent profile of the barrier could be split into several simple curves 28 that could be predicted through the cantilever model. Each of these smaller curves 28 would consist of a single flex sensor 24 as shown in FIG. 10.

For a cantilever beam 26 with a force F at the end, the relationship between max deflection and load is defined by equation 1:

$$δ = - ( {FL}^{3} ) / 3 EI \qquad ( Eq . 1 )$$

where δ is deflection,
F is the force acting on the beam in Newtons (N),
L is length of the beam in meters (m),
E is the modulus of elasticity of the beam material in Pascals (Pa), and
I is the area moment of inertia of the beam's cross section in kilograms/meter² (kg/m²).

Equation 2, below, was obtained through, testing. This equation describes the relationship between the deflection and force. This equation was used to find the Young's modulus of the flex sensor 24 by relating it to equation 1, which was used during predictive modeling.

$$δ = {( 0.9371 {s}^{2} / kg )}^{*} F + 0.0 \; 0215 m \qquad ( Eq . 2 )$$

where s is seconds (s).

Through testing, equation 3, below was developed to determine the relationship between the force and the voltage reported by the flex sensor 24.

$$F = - {( 0.1 \; 589 s / m )}^{*} V + 0.1 \; 458 N \qquad ( Eq . 3 )$$

where V is velocity in m/s.

The force equation (eq. 3) was adjusted based on the baseline voltage (the voltage of the sensor when it is flat), and using the relationship between force and strain in a cantilever beam, the radius that the flex sensor 24 was in was predicted through equation 4, below.

$$ε = - ( y / ρ ) \qquad ( Eq . 4 )$$

where ε is the radius in m.

With an estimation of the stress, strain, and radius of the flex sensor 24, the deformation, and in-plane stresses of the surface on which the sensor is attached can be estimated.

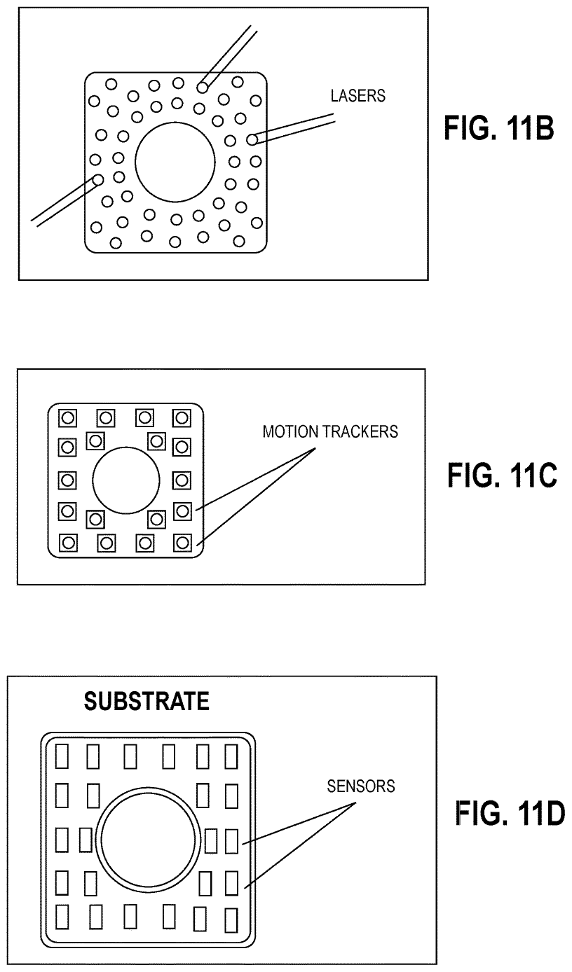

Several concepts for sensing the movement of the barrier and thus the integrity of the seal between the skin barrier and the peristomal skin were generated such as an optical solution, a laser point tracking solution, a motion tracking solution, and the sensor system discussed above, and as schematically illustrated in FIGS. 11A-11D. These concepts were evaluated according to the needs as discussed above, and a solution matrix develop. Each the optical solution (FIG. 11A), the laser point tracking solution (FIG. 11B), the motion tracking solution (FIG. 11C), and the sensor system (FIG. 11D) were found to be acceptable, with the sensor system determined to be preferred, as illustrated in Table 1, below.

TABLE 1
Solution Matrix

| | | | Motion | Sensor |
|---|---|---|---|---|
| | Optical | Laser | Tracking | System |
| 3D Surface map can be | 1 | 1 | 1 | 1 |
| created | | | | |
| Can be used to predict | 1 | 1 | 1 | 1 |
| displacement | | | | |
| Can be used to predict | 0 | 0 | 0 | 1 |
| stress/strain | | | | |
| Attaches to adhesive | 0 | 0 | 1 | 1 |
| barrier | | | | |
| Safe for the ostomate | 1 | 1 | 1 | 1 |
| Does not interfere with | 1 | 1 | 0 | 0 |
| the function of the | | | | |
| ostomy pouching system | | | | |
| Total | 4 | 4 | 4 | 5 |

Several sensors were chosen for testing to determine whether they could be used to predict deformation. The sensors tested include a piezoelectric ribbon sensor, a force sensing resistor, a flex sensor 24, and a conductive sheet.

The piezoelectric sensor 30 was added to a breadboard with a IM resistor across the leads as shown in FIG. 12. The sensor 30 generated a voltage through the piezoelectric effect when it was perturbed. The change in voltage was measured on the nScope relative to ground.

The magnitude of the voltage changes in the resulting scope traces was between 0.1 to 0.5 volts for large movements. Even with the sensitivity maxed out, the change in voltage was transient and difficult to see or to correlate with any specific movement. An alternate circuit configuration incorporating a low pass filter was also tested, but was transient and difficult to see or to correlate with any specific movement. It was observed that the sensor mainly responded to vibrations and not to forces such as bending or tension. The voltage also drifted over a short period of time such that measure of permanent deformations in the barrier would be difficult.

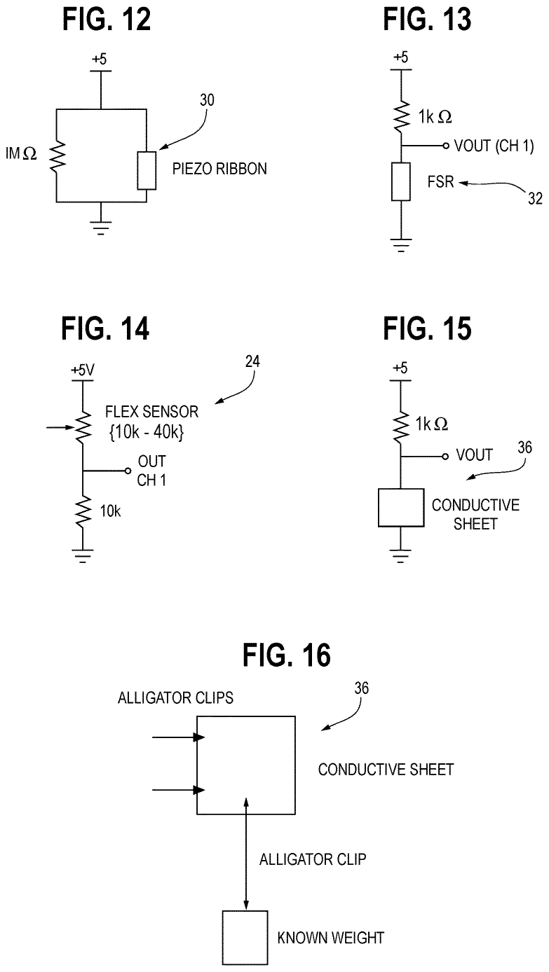

A force sensing resistor was tested in a simple circuit from 5V to ground in a voltage divider configuration as shown in FIG. 13, and the output voltage was shown in the nScope. The force sensing resistor 32 functioned well in detecting the force of compressive loads. The relationship between the compressive force and voltage was clear on the scope trace, and there was no drift for static loads. It was noted, however, that the force sensing resistor 32 did not detect any force during bending, in-plane tension, or in-plane compression. As such, it was determined that this type of sensor can be useful for measuring the adherence of the adhesive barrier to the skin, so it could be usefully incorporated into an AB sensor.

For flex sensor 24 testing, both long and short flex sensors 24 were tested. The flex sensor 24 was set up with a simple circuit from 5V to ground with a series resistor and the scope trace connected to the output of the voltage divider. The configuration of this circuit is shown in FIG. 14.

Resistor values were experimentally determined for each length of the sensor in order to get the best scope traces. The flex sensor 24 functioned well in detecting in-plane bending. The relationship between the voltage reading and the amount the sensor was bent was clear and appeared to be linear. Additionally, the sensor 24 was able to measure bending in both directions. The steady-state unbent voltage hovered around 1V and increased for concave bends, and decreased for convex bends. Because of the flex sensor's 24 ability to measure in-plane bending, it was chosen to be included in further testing. It was characterized according to a cantilever bending model for the final prototype.

For initial testing of the conductive sheet 36, the sheet was connected as one of the resistors in a voltage divider configuration as shown in FIG. 15. The sensor was then subjected to tension, compression, and bending and the voltage change for each of these scenarios was observed.

For the tensile test, the conductive sheet 36 was placed in the voltage divider circuit. It was then placed in uniaxial tension by hanging known masses from it, as illustrated in FIG. 16, and the voltage out value was measured. Equation 5, shown below was used to determine the theoretical strain of the conductive sheet.

$$\begin{matrix}δ = ( IL / EA ) & ε = ( δ / L )\end{matrix} \qquad ( Eq . 5 )$$

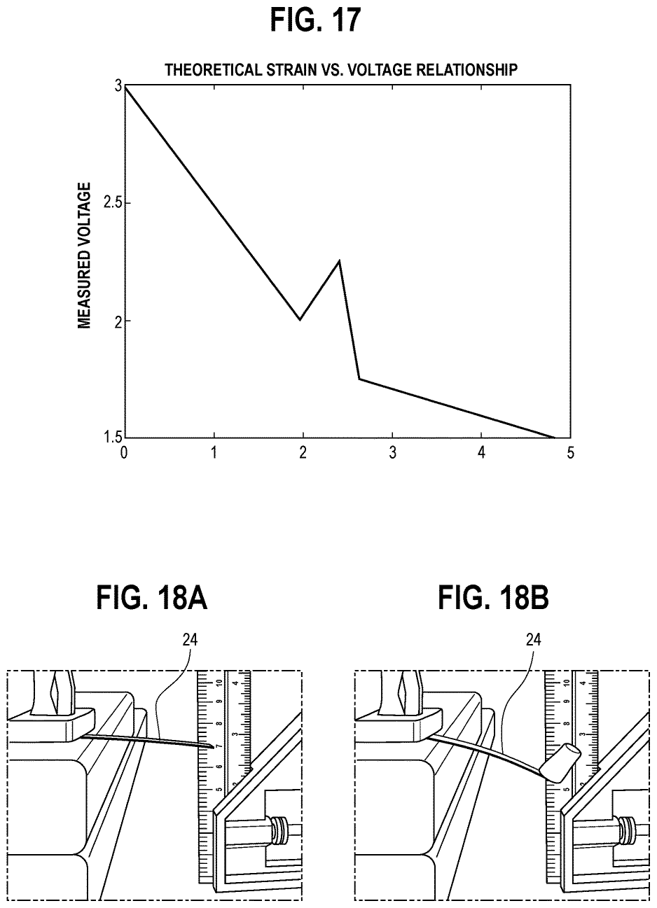

As shown in FIG. 17, the relationship between theoretical strain and measured voltage was unclear. Further testing was conducted to characterize the conductive sheet 36 in tension including additional weights 38 which showed that as the sheet was stretched, the voltage values decreased. Although the conductive sheet 36 could be used to detect tensile forces, it showed low sensitivity and unreliability. Conductive paint testing was also conducted but showed results indicating a lack of a clear relationship between curvature or tension and voltage.

Flex sensor 24 cantilever testing was then carried out and a predictive model developed. A cantilever beam model was used to lead the testing setup and relate experimental data to mathematical equations to facilitate determining the model for chaining multiple sensors together for surface detection.

The sensor 24 was characterized by setting up the sensor 24 in a cantilever configuration with a rigid support as shown in FIG. 18 and placing increasing masses 38 on the end of an anchored flex sensor 24 to simulate different point loads of varying magnitudes. Larger masses were placed on the end of the sensor 24 to represent larger loads. The masses, the voltage of the flex sensor 24, and the height of the tip were measured for each data point. The load and deflection were then calculated from that data.

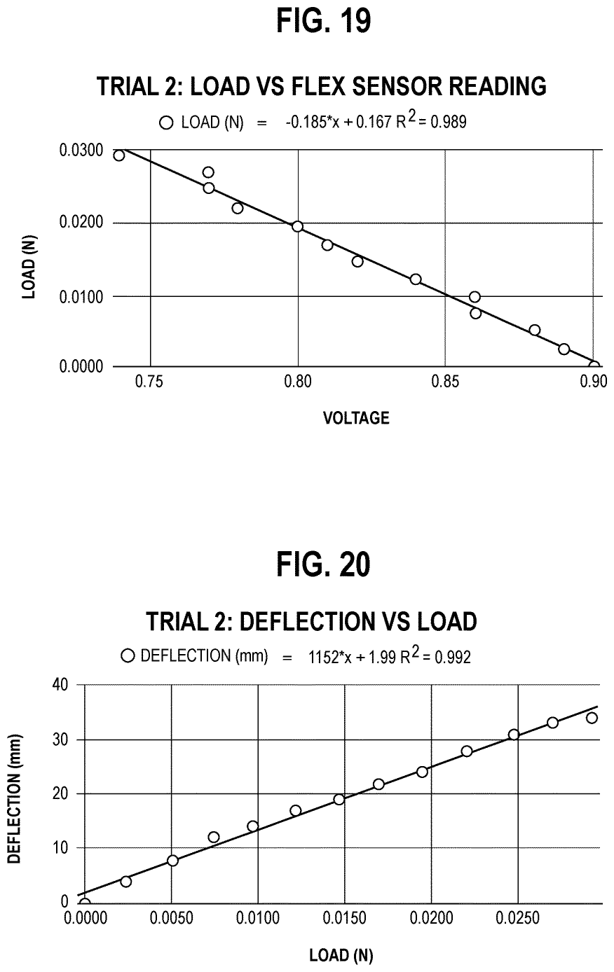

Three trials were conducted for the flex sensor 24 cantilever model. Example plots from the second trial are shown in FIG. 19 which displays the relationship between the load and the flex sensor 24 voltage reading, with an R2 value of 0.989. FIG. 20 shows the relationship between the load and deflection in mm, which was determined with a ruler. The R2 value is 0.992.

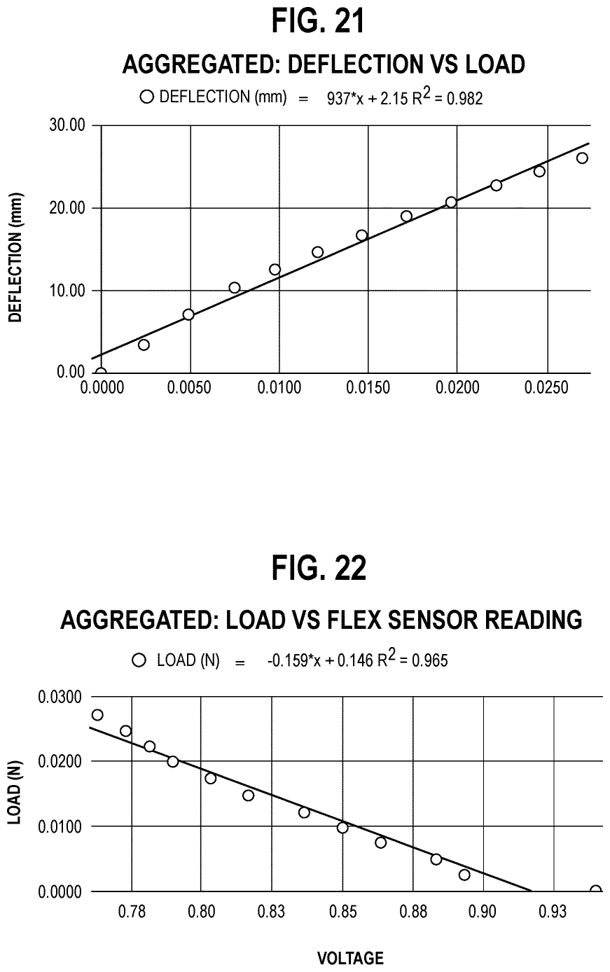

Data from three trials was averaged to form an aggregated model. FIG. 21 illustrates the aggregated data for the relationship between the deflection and load. The R² value of 0.982 suggests that for the flex sensor 24, the cantilever beam model holds. As a result, the cantilever beam model for the flex sensor 24 is as shown in equation 6, below:

$$δ = {( 0.9 \; 371 {s}^{2} / kg )}^{*} F + 0.00215 m \qquad ( Eq . 6 )$$

This was related to the standard deflection equation for cantilever beams with a point load at the end in bending:

$$δ = ( {FL}^{3} ) / 3 EI \qquad ( Eq . 7 )$$

In our developed model, the empirically determined constant (0.9371 s²/kg) is the same as the quantity L³/3EI from the standard equation (Eq. 7). The length of the flex sensor 24 was recorded and the width and height of the flex sensor 24 were used to calculate the moment of inertia, where L=0.541 m and I=5.91×10⁻¹⁴ m⁻⁴.

Using these values and the constant from equation Eq. 2, the Young's modulus can be estimated:

$${L}^{3} / 3 EI = ( 0.9371 {s}^{2} / kg ) \qquad ( Eq . 8 )$$

$$E = 953 MPa$$

This value is of the same order of magnitude that was expected for the polymer casing of the flex sensor 24. A tensile test was unsuccessfully attempted with one flex sensor to try to verify this value, however, testing with multiple sensors 24 should verify the experimentally determined modulus in that we specified that a Young's modulus value of 20 MPa to 200 MPa was desired.

From the data obtained from the cantilever test, the relationship between sensor voltage and applied force was established as shown in FIG. 22. A linear regression was performed on data aggregated from three different trials and was used to characterize the flex sensor. The relationship between sensor voltage and load is:

$$F = - ( 0.1 589 As / m ) * V = 0.1 \; 458 N \qquad ( Eq . 9 )$$

And with a predictive model for force and an estimated Young's Modulus, the moment equation for a cantilever beam can be used to determine the stress and strain of the sensor.

$$M = F ( L - x )$$

$${σ}_{b} = My / I$$

$${σ}_{b} = E ε$$

where F=force applied,
L=total length of the sensor in meters (m),
x=position at which the moment is calculated,
σ_{b}=bending stress in N/m²,
I=moment of inertia,
E=Young's modulus,
y=neutral axis, and
ε=strain.

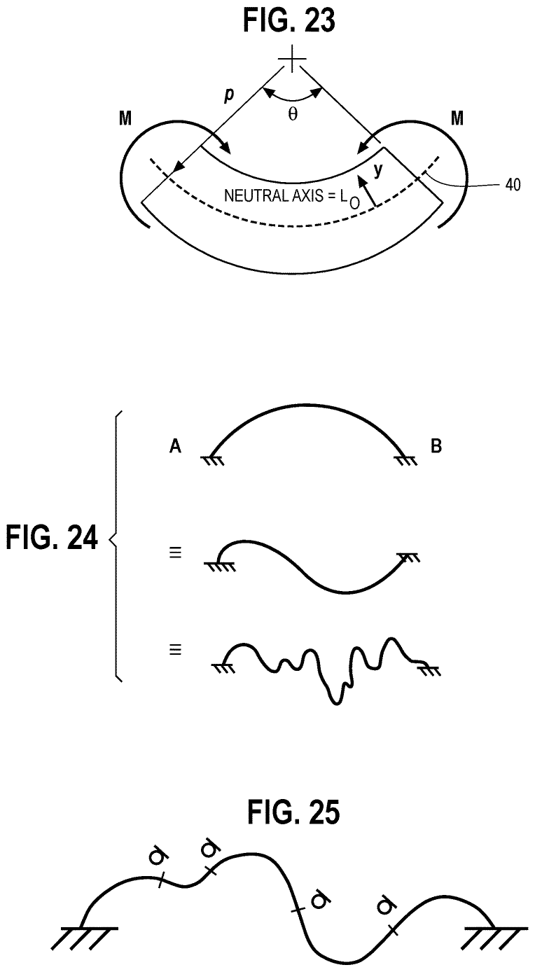

The radius of the flex sensor 24 can be estimated at a certain distance from the neutral axis 40 by using the strain. This relationship is illustrated in FIG. 23, in which p represents the radius at that point in the beam and y represents the neutral axis.

Flex sensors 24 were chained end to end in order to predict more complex shapes. The first critical assumption that was made was that when implemented on the barrier, the flex sensors 24 would be small enough that each of the sensors 24 would only bend as simple shapes that a cantilever model could predict. This is because the flex sensor 24 could display the same voltage value when bent in different shapes as shown in FIG. 24. By implementing this constraint, the shape of the flex sensor 24 can be predicted through the cantilever beam model.

A second critical assumption was that the connection between flex sensors 24 would always be tangent as shown in FIG. 25. This ensures that the flex sensor 24 chain could predict the shape of a curve without having a concern of sharp corners between sensors 24.

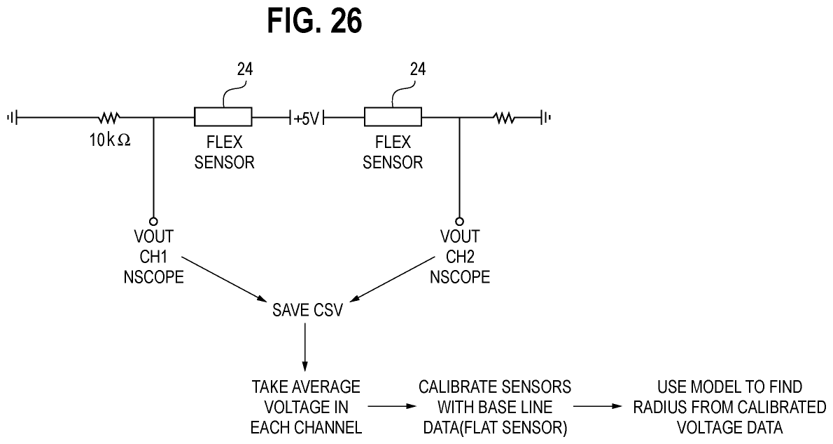

Sensor substrate selection consisted of testing three different substrates to determine which would allow us to embed or attach the flex sensors 24 without interfering with data acquisition. It was found that sandwiching the sensors between tape worked best in that the flex sensor 24 was able to deform correctly to the shape being measured and the sensor's 24 behavior was not altered. FIG. 26 shows the electrical diagram used for the critical system prototype. As illustrated, both flex sensors 24 were connected as part of a voltage divider that goes from +5 to ground. The output of each of these voltage dividers was connected to CH1 and CH2 of the nScope that captures a trace of the voltage measured depending on how much the flex sensor 24 is bent. This data was then saved as a csv file and the voltage output for each sensor 24 is averaged. The math model is calibrated based on the baseline of each sensor 24 and the voltage collected is used to find predicted force and radius measured by the sensor 24.

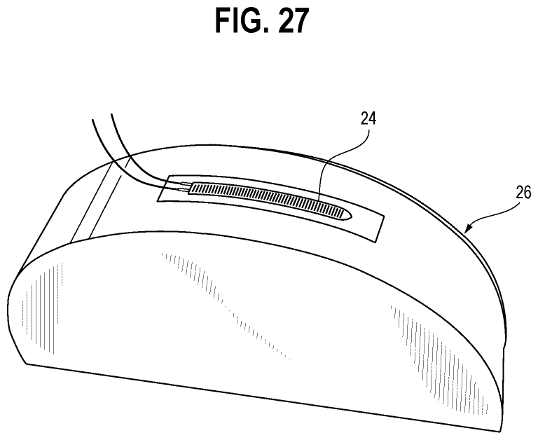

Predictive modeling was then made using single sensor 24 without substrate. The cantilever beam predictive model was first validated by wrapping the flex sensor 24 around known radii and comparing the predicted radius to the actual radius as seen in FIG. 27 which illustrates the setup for this testing with the flex sensor 24 being wrapped around a 3D printed test bench 26.

The baseline voltage for the sensor 24 being tested was first measured and the force equation was adjusted to account for this by plugging in the value into the original equation and adding the result to the force equation. Then, the voltage when the sensor 24 was bent around a known radius was calculated and plugged into the modified force equation to find the predicted force. This force was used to find the predicted moment, stress, strain, and radius along the length of the flex sensor. An example of this testing is shown in Table 2, below.

| Position | Moment | Stress | | Radius |
|---|---|---|---|---|
| (mm) | (N*m) | (MPa) | Strain | (mm) |
| 0 | 1.46E−3 | 5.93 | 6.23E−3 | 77.08 |
| 27.05 | 1.39E−3 | 5.64 | 5.92E−3 | 81.14 |
| 54.10 | 1.32E−3 | 5.34 | 5.60E−3 | 85.64 |
| 81.15 | 1.24E−3 | 5.04 | 5.29E−3 | 90.68 |
| 10.82 | 1.17E−3 | 4.75 | 4.98E−3 | 96.35 |
| 13.53 | 1.10E−3 | 4.45 | 4.67E−3 | 102.77 |
| 1.623 | 1.02E−3 | 4.15 | 4.36E−3 | 110.11 |
| 18.94 | 9.50E−4 | 3.86 | 4.05E−3 | 118.58 |
| 21.64 | 8.77E−4 | 3.56 | 3.74E−3 | 128.47 |
| 24.35 | 8.04E−4 | 3.26 | 3.43E−3 | 140.14 |
| 27.05 | 7.31E−4 | 2.97 | 3.11E−3 | 154.16 |

In this testing the baseline voltage was 0.77 V, the voltage when the sensor 24 was bent was 0.6 V, and the predicted force was 0.027 N. The actual radius of the object was 77 mm and the closest radius value predicted by the model was 77.08 mm as shown in Table 2, which occurred at what would be the support of the cantilever beam. This was consistent along all radii tested, and was thus the value that most closely predicts the radius being measured.

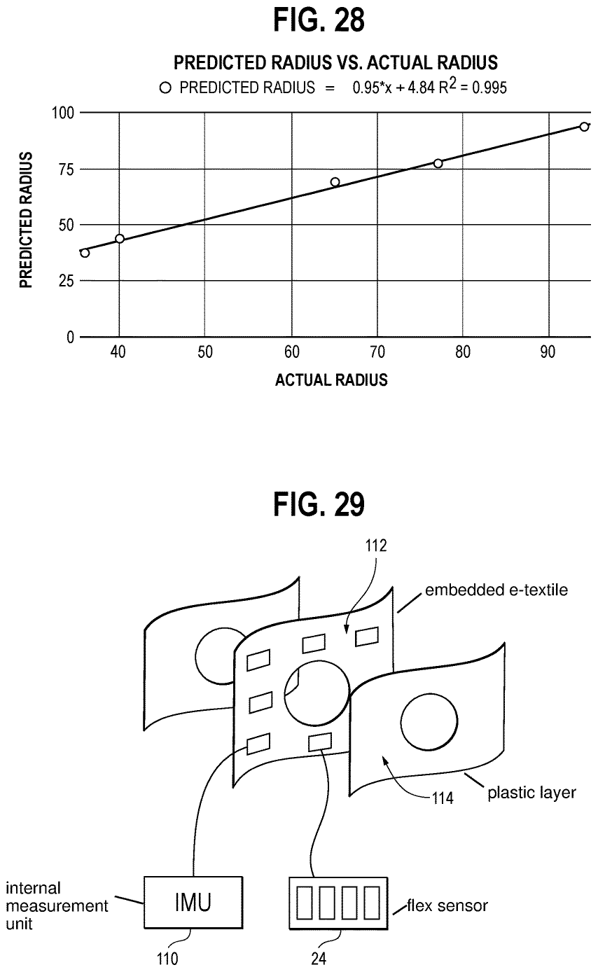

The process was repeated for various radii and the actual value was compared to the predicted value as shown in FIG. 28 and table 3, below.

TABLE 3
Actual vs. Predicted Radius

| Actual | Predicted |
|---|---|
| Radius | Radius |
| 36.05 | 37.44 |
| 40 | 43.68 |
| 65.09 | 69 |
| 77 | 77.08 |
| 94 | 93.6 |

The R₂ value for this relationship was 0.995 which confirms that the predictive model works to predict the radius from the voltage measured for a single flex sensor 24 without a substrate. According to the needs and metrics, the correlation between the predictive model and the actual values should have an R² value of at least 0.7 and ideally of 0.95.

A similar approach was taken to validate the chained flex sensor 29 model. For this, the chain of flex sensors 29 was placed flat on the table and the baseline voltages were recorded. The predictive force equation was modified for each sensor based on each sensor's baseline voltage. Then, the chain of sensors 29 was wrapped around a test bench 26 with a radius of 94 mm and the voltage recorded by the nScope on a csv file was averaged.

The data obtained from two trials is shown in Table 4, below.

TABLE 4
Actual vs. Predicted Radius For Chained Sensors

| | | | Bent | Predicted |
|---|---|---|---|---|
| Trial | Sensor | Baseline | Voltage | Radius |
| 1 | 1 | 1.37 | 1.22 | 91.95 |
| 1 | 2 | 1.50 | 1.36 | 92.29 |
| 2 | 1 | 1.29 | 1.15 | 91.63 |
| 2 | 2 | 1.28 | 1.13 | 86.21 |

From testing this configuration we observed that this configuration was less accurate in measuring the actual radius than the single flex sensor without any substrate. We further observed that the baseline voltage changed significantly from trial to trial in this configuration which may have been what caused the model to be less accurate. It is believed that the baseline changed frequently due to the wires that attached to the flex sensor being stiff and which can cause the sensor to wrap around which is more difficult to control when the sensors are in a chain configuration. It is envisioned that less stiff sensors could be determine whether predictions are improved. Other adjustments could be made to the predictive model for a flex sensor chain 29 wrapped around tape to make the model more accurate for this scenario.

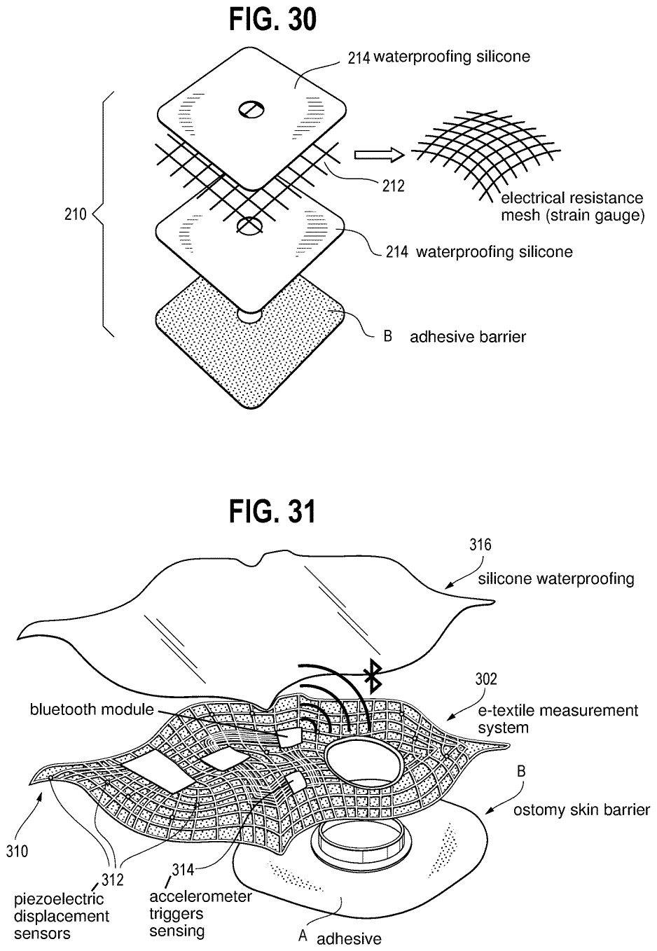

Other configurations of peristomal skin barrier sensors were contemplated. For example, FIG. 29 illustrates an inertial measurement unit 110 in an embedded e-textile 112 with flex sensors 24 in the textile. A plastic layer 114 is positioned on the e-textile 112. FIG. 30 illustrates an electrical resistance mesh sandwich 210 having an electrical resistance mesh/strain gauge 212 sandwiched between waterproofing silicone layers 214 and is positioned on an adhesive barrier B. The electrical resistance mesh/strain gauge 212 uses the change in resistance to generate a 3 dimensional surface model.

FIG. 31 illustrates an e-textile measurement system 302 having an e-textile 310 with a piezo mesh. The e-textile 310 includes piezo displacement sensors 312 and can use one or more accelerometers 314 to trigger sensing. The e-textile is sandwiched between the ostomy skin barrier and a silicone waterproofing layer. The e-textile can be secured to the barrier B by an adhesive A. A silicone or other waterproof layer 316 can be positioned on the e-textile 310.

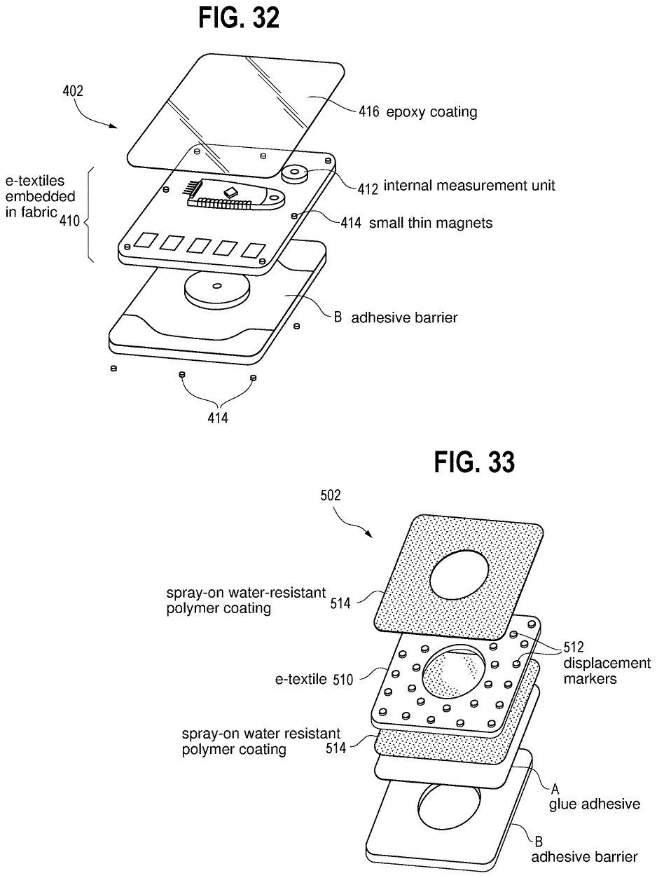

FIG. 32 illustrates an embodiment of a measurement system 402 in which an e-textile 410 includes an inertial measurement unit 412 with small thin magnets 414 and an epoxy coating layer 416 on the e-textile, with the e-textile 410 sandwiched between the epoxy layer 416 and the adhesive barrier B. The adhesive barrier B can likewise include small thin magnets 414 to maintain the e-textile 410 on the adhesive barrier B.

FIG. 33 illustrates yet another embodiment of a measurement system 502 in which the e-textile 510 having displacement markers 512 is sandwiched between water resistant polymer coating layers 514 (that can be sprayed onto the e-textile 510), and a glue adhesive A between the water resistant polymer coating 514 and the adhesive barrier B.

It will be appreciated that the presently disclosed peristomal skin barrier sensor measures the deformation of the barrier to measure the movement of the adhesive barrier so as to gain an understanding of the changes the barrier undergoes in use. Such a system senses the deformation and movement of the adhesive barrier. Such a sensor system includes an internal measurement unit (IMU) and a sensor, which sensor is configured to measure a deformation of the barrier.

All patents referred to herein, are hereby incorporated herein in their entirety, by reference, whether or not specifically indicated as such within the text of this disclosure. In addition, it is understood that terminology referring to directions or relative orientations, such as, but not limited to, “forward” “rearward” “inner” “outer” “upper” “lower” “raised” “lowered” “top” “bottom” “above” “below” “alongside” “left” and “right” are used for purposes of example and do not limit the scope of the subject matter described herein to such orientations or relative positioning.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A peristomal skin barrier sensor comprising:
a barrier;
an internal measurement unit (IMU); and
a flex sensor, wherein the flex sensor is configured to measure a deformation of the barrier.

2. The peristomal skin barrier sensor of claim 1, wherein the flex sensor is a series of flex sensors.

3. The peristomal skin barrier sensor of claim 2, wherein the series of flex sensors are chained to adjacent ones of the flex sensors.

4. The peristomal skin barrier sensor of claim 3, wherein data is collected at each of the flex sensors of the series of flex sensors.

5. The peristomal skin barrier sensor of claim 4 wherein the data collected is voltage data.

6. The peristomal skin barrier sensor of claim 1, wherein the flex sensor is configured to test a structure of the barrier.

7. A peristomal skin barrier sensor comprising:
a barrier;
an electrical resistance mesh, wherein the electrical resistance mesh is configured to measure a resistance used to generate a 3D surface model; and
a waterproof silicone layer.

8. The peristomal skin barrier sensor of claim 7, wherein the waterproof silicone layer is a first waterproof silicone layer and wherein the electrical resistance mesh is sandwiched between the first waterproof silicone layer and a second waterproof silicone layer.

9. The peristomal skin barrier sensor of claim 8, wherein one of the first and second silicone waterproof layers is positioned adjacent the barrier.

10. The peristomal skin barrier sensor of claim 7, wherein the electrical resistance mesh is configured to test a structure of the barrier.

11. A peristomal skin barrier sensor comprising:
a barrier;
a e-textile measurement system, wherein the e-textile measurement system comprises an e-textile and one or more piezoelectric displacement sensors configured to measure a deformation of the barrier; and
a silicon waterproof layer.

12. The peristomal skin barrier sensor of claim 11, further including an accelerometer on the e-textile.

13. The peristomal skin barrier sensor of claim 12, wherein the accelerometer triggers the one or more piezoelectric displacement sensors.

14. The peristomal skin barrier sensor of claim 11, wherein the e-textile measurement system is configured to test a structure of the barrier.

15. The peristomal skin barrier sensor of claim 11, wherein the e-textile is positioned between the silicone waterproof layer and the barrier.

16-19. (canceled)
